(19) United States
(12) Patent Application Publication
(10) Pub. No.: US 2019/0276767 A1
(43) Pub. Date: Sep. 12, 2019

(54) CANNABIS EXTRACT FILTRATION METHODS AND SYSTEMS

(71) Applicant: PURE, LLC, Portland, OR (US)

(72) Inventor: Kevin Jered HOUNSHELL, Portland, OR (US)

(21) Appl. No.: 16/421,132

(22) Filed: May 23, 2019

Related U.S. Application Data
(62) Division of application No. 16/017,674, filed on Jun. 25, 2018.
(60) Provisional application No. 62/523,897, filed on Jun. 23, 2017.

(51) Int. Cl. C11B 3/00; B01D 15/22; C11B 3/10

(52) U.S. Cl. CPC C11B 3/008; C11B 3/10; C11B 3/001; B01D 15/22

(57) ABSTRACT

Methods and systems for filtering a plant-derived extract to remove contaminants, including a filter column containing a filter medium and a vacuum pump. The plant-derived extract is mixed with a solvent, and drawn through the filter column across the filter medium. In various embodiments, the solvent and filter medium may be selected to target specific contaminants or classes of contaminants, such as herbicides, insecticides, fungicides and/or other synthetic agrichemicals.

### CROSS-REFERENCE TO RELATED APPLICATIONS

This application is a divisional of U.S. patent application Ser. No. 16/017,674, filed Jun. 25, 2018, which claims priority to U.S. Provisional Application No. 62/523,897, filed Jun. 23, 2017, the entire disclosures of each application are incorporated herein by reference.

### TECHNICAL FIELD

Embodiments described herein generally relate to methods and systems for filtration for plant-derived extracts, and more particularly, to methods and systems for filtering impurities from cannabis extracts such as cannabis oil.

### BACKGROUND

A significant portion of emerging legal cannabis markets are focused on cannabinoid extracts and concentrates. These popular products with high cannabinoid potency include CO₂-extracted cannabis oil, distillate, shatter, and live resin. However, the prevalent use of insecticides, fungicides, herbicides, and other chemicals on cannabis crops frequently results in those compounds becoming concentrated during the extraction process, and extracted along with the cannabinoids, terpenes, and flavonoids which are the desirable targets of extraction.

As cannabis is legalized in various jurisdictions, regulators impose purity standards and associated testing requirements to ensure quality and the safety of cannabis products. The presence of pesticides and other horticultural chemicals in finished cannabis extracts poses health risks to consumers and can prevent products from passing these regulatory compliance screening for pesticide residue.

### BRIEF DESCRIPTION OF THE DRAWINGS

FIG. 1 depicts a filtration system for cannabis oil, according to various embodiments.

FIG. 2 depicts the filter column and filter media of the filtration system of FIG. 1, according to various embodiments.

FIGS. 3A and 3B depict the fitting for the first end of the filter column depicted in FIG. 2, according to various embodiments.

FIGS. 4A and 4B depict the fitting for the second end of the filter column depicted in FIG. 2, according to various embodiments.

FIGS. 5A, 5B, and 5C depict the arrangement of components connecting the second end of the filter column of FIG. 2 with a collection reservoir and vacuum pump, according to various embodiments.

FIG. 6 is a flowchart of a method for employing the filtration system of FIG. 1, according to various embodiments.

### DESCRIPTION OF EMBODIMENTS

When cannabis or other plant extract products do not meet regulatory quality standards and/or are found to be laced with undesirable, potentially harmful contaminants, it may require that the products and source batches be discarded, resulting in loss of revenue. Alternatively, growers may forego the use of agrichemicals such as herbicides, insecticides, fungicides, and the similar such products, but potentially at the expense of diminished crop yields due to problems that the agrichemicals would have otherwise prevented or suppressed. Thus, the ability to either prevent or remove contamination of such agrichemicals from plant extract products obtained from treated crops may enable growers to use effective agricultural tools to maximize crop yields, while providing finished products that are safe for consumption or subsequent use and/or meet any applicable regulatory standards.

The various embodiments described herein facilitate the removal of unwanted compounds from plant-derived liquids, solids, and semisolids. Embodiments of the disclosed cannabis filtration system selectively remove various undesirable and/or harmful substances, such as pesticides, herbicides, fungicides, artificial fertilizers, and other agricultural-related products from plant-derived extracted products using a unique liquid-solid extraction technique. In some embodiments, removal is effected by using a combination of a resin, resin blend, or other similar medium as a filtration medium in combination with appropriate solvents. In some embodiments, de-waxed plant-derived products are diluted with an appropriate organic solvent, which may be chosen based on the compounds believed to be present in the sample or based on the physical state of the sample, in a ratio ranging from approximately 1:1 up to 10:1 solvent to sample dilution. Mixtures outside of this range may result, depending upon system configuration, may begin to exhibit increasing inefficiency, to the point of failing to work. Plant-derived products may be further purified by removing non-naturally occurring impurities that would be not normally be found within the plant.

In the following detailed description, reference is made to the accompanying figures which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements are in direct physical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

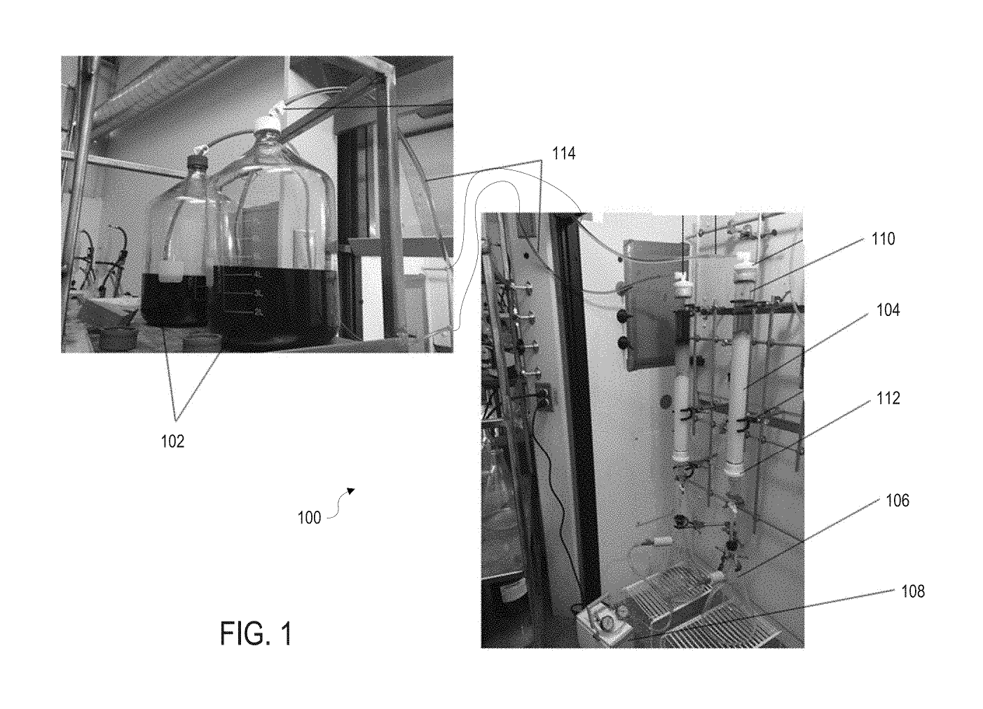

In FIG. 1, the various components of filtration system 100 are depicted. System 100 includes a solution reservoir 102 which is in fluid communication with a first end 110 of filter column 104. Filter column 104 in turn is in fluid communication with a collection reservoir 106, which is secured to a second end 112, opposing the first end 110. A vacuum pump 108 is further in fluid communication with filter column 104 on second end 112, so as to create a low pressure side with collection reservoir 106.

Solution reservoir 102 may be any suitable container that is non-reactive with and will not otherwise contaminate or taint the plant-derived extract or solvent with which the plant-derived extract may be mixed. In one embodiment, solution reservoir 102 comprises one or more ten-liter borosilicate glass media bottles, also known as carboys. Other sizes may be employed depending upon the amount of extract to be processed and/or the overall capacity and throughput of system 100. Extract-solvent mixture may be removed from solution reservoir 102 from any suitable location on solution reservoir 102, e.g. via a lid or cap, a fitting, a tap, an outlet, or a similar type of fixture. For example, solution reservoir 102 may be equipped with a cap upon its top configured with a dip tube to allow conveying the extract-solvent mixture to filter column 104. In another example, solution reservoir 102 may be configured with a tap or outlet near the bottom of the reservoir, to allow extract-solvent mixture to feed via gravity.

Solvent reservoir 102 is in fluid communication with filter column 104. In some embodiments, solvent reservoir 102 may be fitted directly upon filter column 104, and solvent reservoir 102 may include an outlet near its bottom, which can act to allow the extract-solvent mixture to drain directly into first end 110 of filter column 104 by action of gravity. In other embodiments, solvent reservoir 102 may be in fluid communication with filter column 104 by a hose or tubing 114, which may conduct the extract-solvent mixture to filter column 104. In embodiments, the extract-solvent mixture may be carried from solvent reservoir 102 to filter column 104 by action of gravity, by a pumping pressure (either by a positive pressure within solvent reservoir 102, a negative pressure through filter column 104, or both), or by a combination of gravity and pressure.

According to one particular embodiment, each solution reservoir 102 includes a cap which screws onto a threaded neck of the reservoir 102. Each cap has a hole drilled in it to allow % in-diameter vacuum tubing 114 to be fed through to the interior of solution reservoir 102, to place it in fluid communication with the extract-solvent mixture. The tubing 114 may be equipped with plastic tubing clamps to control flow within the vacuum tubing 114. Tubing 114 in turn connects to first end 110 of filter column 104. In the embodiment, to ensure a desirable flow rate, the solution reservoir 102 is elevated above the column, thereby allowing transportation of the extract-solvent mixture into filter column 104 by both negative pressure provided by vacuum pump 108 and gravity assistance.

Tubing 114 may be manufactured from any material suitable to the nature of the extract-solution. In some embodiments, tubing 114 may be manufactured from a relatively insert plastic, such as vinyl. In other embodiments, tubing 114 may be manufactured from a metal, such as stainless steel. Other embodiments may use another suitable material. Tubing 114 is sized depending upon the particular details of a given implementation of system 100.

Extract-solvent mixture passes from solution reservoir 102 into first end 110 of filter column 104, through the column, and then to second end 112 of filter column 104, where it drains into a collection reservoir 106. In some embodiments, and depending upon the nature of the extract-solvent mixture and medium used in filter column 104, gravity may be sufficient to ensure an appropriate flow through system 100 into collection reservoir 106. In other embodiments, a vacuum pump 108 may supply the motive force necessary to move the extract-solvent mixture through filter column 104, either on its own or with gravity assistance. As with a gravity feed implementation, whether a vacuum pump 108 is used in conjunction with gravity, or is used entirely by itself (such as where system 100 is configured with its various components approximately horizontal), may depend upon the nature of the extract-solvent mixture and medium used in filter column 104.

In embodiments employing vacuum pump 108 for some or all of the force necessary to move the extract-solvent mixture through system 100, vacuum pump 108 may be attached on the second end 112 side of filter column 104. In some embodiments, vacuum pump 108 may connect to a fitting or nipple on collection reservoir 106. In other embodiments, vacuum pump 108 may connect to an intermediate fitting or tap between second end 112 and collection reservoir 106. Any configuration that allows vacuum pump 108 to create a low pressure on the second end 112 side of filter column 104 to supply a force to move the extract-solvent mixture from first end 110 side to second end 112 side and into the collection reservoir 106 may be employed.

As with solution reservoir 102, collection reservoir 106 may be any suitable container that is non-reactive with the extract-solvent mixture. In various embodiments of system 100 that use a vacuum pump 108 to create a negative pressure on second end 112 and into collection reservoir 106, collection reservoir 106 should be able to withstand the low pressure generated by vacuum pump 108. As described above, collection reservoir 106 may include a fitting or nipple to allow vacuum pump 108 to be attached, to create a low pressure on the second end 112 side. Collection reservoir 106 further is configured to be in fluid communication with second end 112 to receive extract-solvent mixture that has passed through filter column 104. Such communication may be accomplished by a fitting or stopper that allows extract-solvent mixture to pass into collection reservoir 106. As with solution reservoir 102, collection reservoir 106 may directly connect to filter column 104, or may be connected by way of tubing, piping, or other suitable fluid channels.

One or more collection reservoirs 106 collect the filtered solution and are connected to both the filter column 104 and the vacuum pump 108. In some embodiments, collection reservoir 106 may be a vacuum filter flask. For example, depending upon the quantity of extract to be purified and/or the throughput of filter column 104, collection reservoir 106 may be a 5-liter flask; in another example, a 3-liter flask may have sufficient capacity and be so employed. In other embodiments, multiple collection reservoirs 106 may be attached to a single filter column 104. The volume of a given collection reservoir 106 or series of collection reservoirs 106 may be sized with respect to the size of solution reservoir 102.

Vacuum pump 108 may be any pump capable of pulling at least a partial vacuum. In embodiments, vacuum pump 108 may be specifically designed for laboratory or industrial vacuum filtration applications. A variety of brands and models of laboratory vacuum pumps may be suitable for system 100. In one embodiment depicted, vacuum pump 108 is an oil-free piston pump with a maximum pump capacity of 26 liters/minute and the ability to draw a vacuum down to 100 Torr. Other embodiments may use a pump 108 with different specifications, which may be determined at least in part by the characteristics of the extract-solvent mixture, the filter medium used in filter column 104, the size of system 100 and/or the desired throughput. Vacuum pump 108 may be equipped with a single or multiple taps for drawing a vacuum. In embodiments using a vacuum pump 108 that includes multiple taps, a single vacuum pump 108 may be useable with multiple iterations of system 100. Vacuum pump 108, or another suitable point in system 100, may be equipped with a vacuum gauge or another similar device to monitor the performance of system 100.

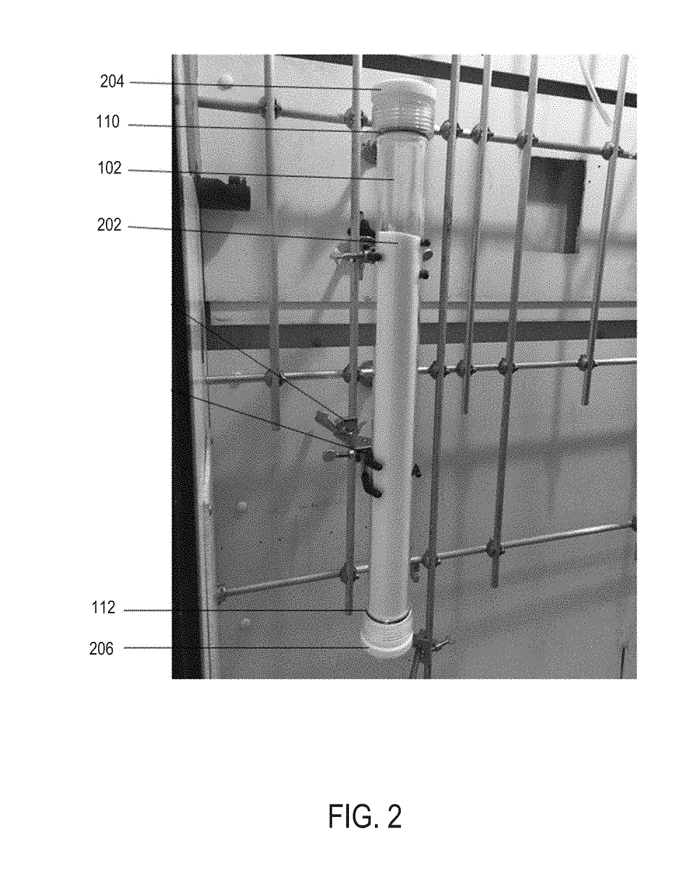

Referring to FIG. 2, in the depicted embodiment filter column 104 is an open-ended glass cylinder with internal threads (#50) on each end. The column's inner diameter is 50 mm and its inner length is 600 mm; these measurements do not include the threaded ends. The first end 110 of the column is sealed with a first fitting 204, which in one particular embodiment is a threaded PTFE adapter with #50 threads. The second end 112 is sealed with a second fitting 206, which in the embodiment is a threaded PTFE reducing coupling with 50/25 thread. Before seating either the first fitting 204 or the second fitting 206, in the depicted embodiment a 50 mm-diameter circular filter is placed inside each component and an FETFE O-ring (size 136) is placed at the end of each component, just beyond the threads. The filters may have a rough side and a smooth side; the rough side faces the column interior. For the depicted embodiment, once the second fitting 206 is in place, approximately 0.5 kg of dry media 202 is poured in through the open top of the column.

Although filter column 104 is manufactured from glass in the depicted embodiment, filter column 104 may be manufactured from other suitable materials, such as stainless steel or another relatively inert metal, plastic, or any other suitable material that is non-reactive or otherwise compatible with the extract-solvent mixture and the filter media 202.

Filter column 104 is substantially filled with a filter medium 202, which may be selected with respect to a particular contaminant or contaminants that are targeted for removal, the nature of the particular plant-derived extract and/or the type of solvent mixed with the extract. In one embodiment, the filtration media 202 is a crystalline structured resin. The actual composition of media 202 may vary for each compound that is the target of the extraction. Example media 202 may include straight chain silica bonded (i.e. C8 media), reverse phase resin, de-ionization resins, Florisil® (brand name for activated/synthetic magnesium silicate), activated carbon, and silica gel. Filtration media 202 may be specific to the impurities it removes. For example, de-ionization resin can remove metal contaminants, Florisil® can be used to remove pesticide impurities, and activated carbon is useful to remove color impurities. The choice of filter medium 202 may impact the choice of solvent used to create the extract-solvent mixture, with the efficacy of certain filtration media 202 impacted (e.g. enhanced or facilitated) by the choice of solvent. In the filter column 104 in the depicted embodiment, approximately 0.5 kg of media is used to fill each filter column 104.

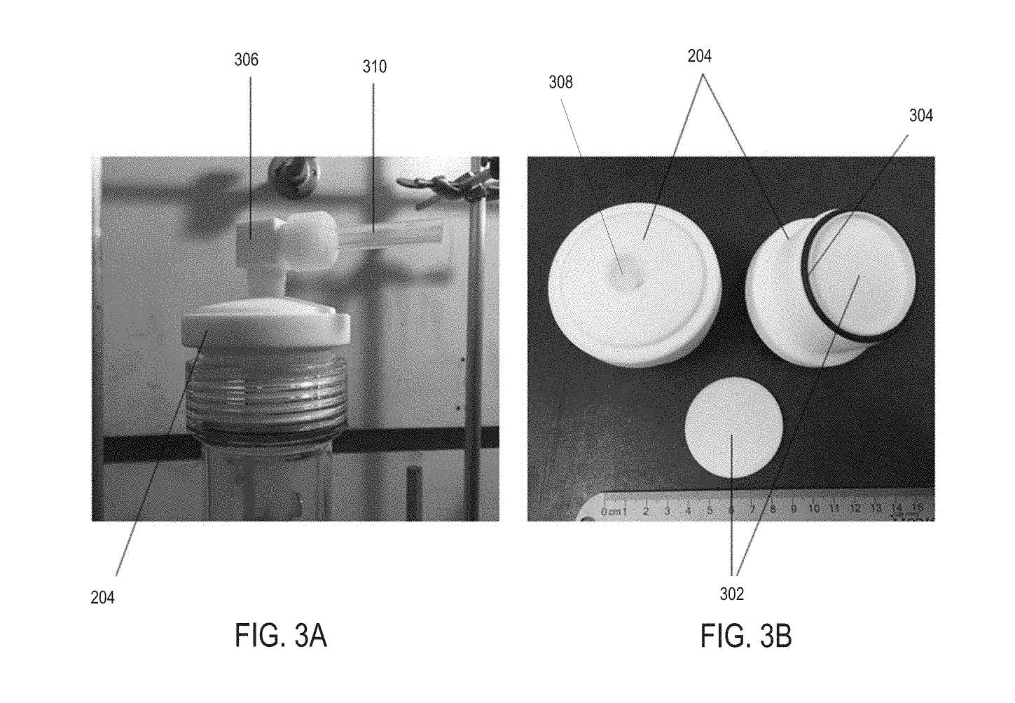

FIGS. 3A and 3B depict first fitting 204, which is secured to first end 110 of filter column 104. In some embodiments, first fitting 204 is manufactured from a non-reactive material, and is cylindrical in design. The bottom (not labeled in the figures, the side of first fitting 204 that faces the interior of filter column 104) may contain a disc-shaped filter disc 302. Filter disc 302 may be constructed from a PTFE (Teflon) material, nylon, or another non-reactive material that may be the same or different from the material of first fitting 204. Filter disc 302 is configured to allow an extract-solvent mixture to pass through relatively unimpeded. In some configurations, filter disc 302 may have pore sizes that are approximately 20 μm in diameter. Other sizes may be used as appropriate to the nature of the extract-solvent mixture. In at least one embodiment, the pore size must be at least small enough to prevent particles of filter medium 202 from escaping, while large enough within this constraint to ensure the extract-solvent mixture can pass through. Filter disc 302 may further act to prevent solid contaminants that may be suspended or picked up in the extract-solvent mixture from being introduced to and potentially interfering with filter medium 202. Filter disc 302 may also serve to prevent filter medium 202 from escaping from filter column 104 into tubing 114 and/or solution reservoir 102.

In the depicted embodiment in FIG. 3B, the end of first fitting 204 is fitted with a size 136 FETFE O-ring 304. O-ring 304 seats against the first end 110 of filter column 104, to provide a water-tight (and potentially air-tight) seal, so as to prevent leakage of the extract-solvent mixture. Other embodiments may omit the O-ring 304, manufacture O-ring 304 from a different material suitable to the nature of the extract-solvent mixture, or use a different type or configuration of seal. With first fitting 204 secured and seated on the column 104, in the depicted embodiment a PTFE tube compression elbow 306 (⅜ npt) is threaded into an orifice 308 opening on the first fitting 204. A 75-100 mm segment 310 of ⅜-in PTFE tubing is inserted horizontally into the elbow's 306 opening and secured in place with the elbow's 306 nut, which may be made of PVDF. Tubing 114 may in turn be secured to segment 310. In other embodiments, solution reservoir 102 may be directly secured to orifice 308. In still other embodiments, a different elbow or connector may be secured to first fitting 204, depending upon the requirements of a particular implementation of system 100.

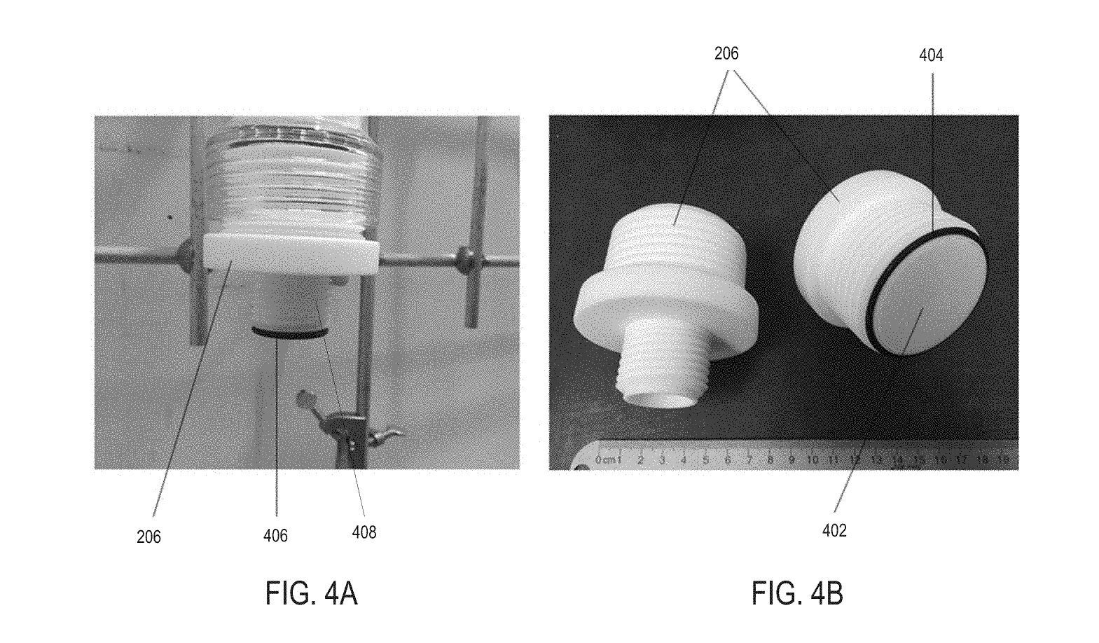

FIGS. 4A and 4B depict second fitting 206, which is secured to second end 112 of filter column 104. Second fitting 206 may be constructed from similar materials as first fitting 204. Second fitting 206, in the depicted embodiment, has a bottom coupling 406 that may be sized differently from the body of second fitting 206. The size of bottom coupling 406, as with the body of second fitting 206, may depend upon the specifics of a given implementation of system 100. In some embodiments, second fitting 206 may include a filter disc 402 and/or an O-ring 402 which seats against the second end 112 of filter column 104. Filter disc 402 may be of comparable construction to filter disc 302, and O-ring 404 may be of similar construction to O-ring 304. Filter disc 402 may act to retain filter medium 202 within filter column 104, and in some embodiments may be configured with different pore sizes compared to filter disc 302 suitable to helping to retain filter medium 202. In the depicted embodiment, the bottom coupling 408 of second fitting 206 receives a size 121 FETFE O-ring 406 just below its threads, to seal with an adapter (described below).

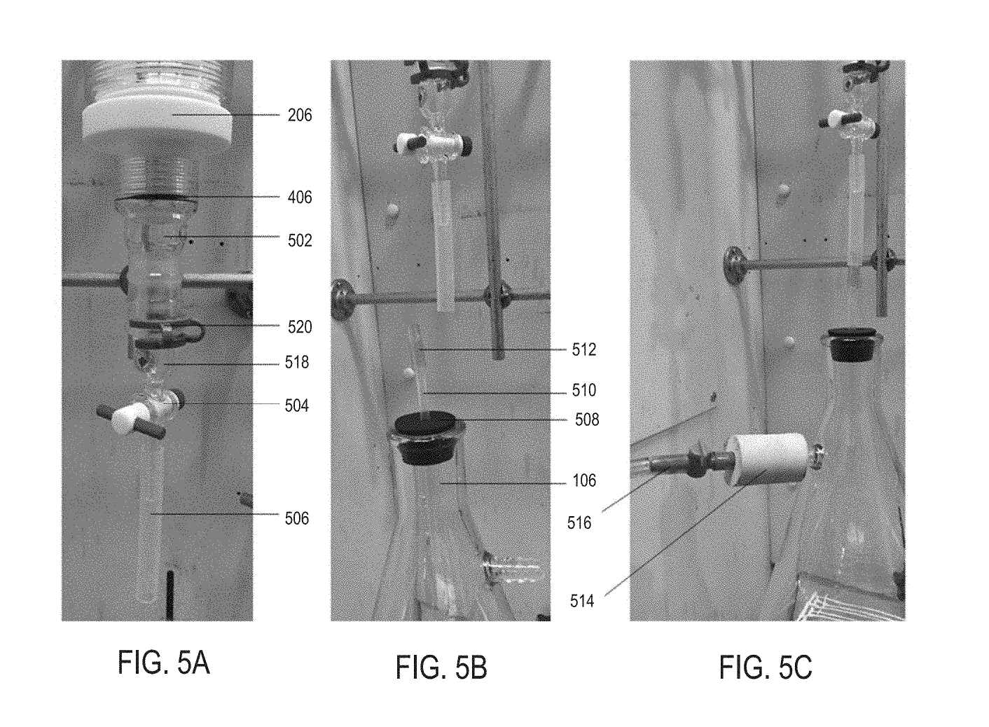

FIGS. 5A-5C depict the arrangement of components for the connection between second end 112 of filter column 104 with the collection reservoir 106. In some embodiments, a conversion adapter 502 is threaded on to the bottom coupling 408 of second fitting 206. In the depicted embodiment of FIG. 5A, conversion adapter 502 is a 24/40 #25 glass conversion adapter. Other embodiments may use a different size conversion adapter 502, or may omit the conversion adapter 502. Still other embodiments may include a second adapter 518 that is interposed between adapter 502 and stopcock 504. Conversion adapter 502 may be secured to second adapter 518 by a clip 520 or other suitable connection means. Adapters 502 and 518 may interface by any known mechanism, e.g. a frosted glass portion that can be treated with high vacuum grease. The conversion adapter 502 may include a stopcock 504, which in turn may have a 60-100 mm segment 506 of ⅜ in PTFE tubing covering its base, as in the depicted embodiment. Stopcock 504 may be used in some embodiments to control the flow of filtered extract-solvent mixture into collection reservoir 106.

When only one collection reservoir 106 is used in an implementation of system 100, stopcock 504 (which may have an HDPE body and a polypropylene tap) may be used to control the flow of extract-solvent mixture through system 100. Where more than one collection reservoir 106 is employed and each reservoir 106 is connected to a common filter column 104 (and potentially a common vacuum pump 108), a 3-way stopcock 504 may be used to facilitate directing the flow of extract-solvent mixture from the column 104 into a second (or third) collection reservoir 106, once the preceding collection reservoir 106 reaches capacity.

FIG. 5B depicts the arrangement of components for connection of a collection reservoir 106 to second end 112 of filter column 104, according to a possible embodiment. Collection reservoir 106 may be sealed with a fitting 508 that is suitable to the particular implementation of collection reservoir 106. In the depicted embodiment, fitting 508 is a rubber stopper (e.g. size #6 or #8½) with an orifice to allow insertion of a tubing 510. In some embodiments, tubing 510 may comprise a segment of PTFE tubing with a ¼-in outer diameter. In other embodiments, tubing 510 may be constructed from materials similar to 114. In some embodiments, the top of the PTFE tubing 510 may be covered with a short segment 512 of ¼-in vacuum tubing, to ensure good fit with the ⅜-in PTFE tubing segment 506 that extends down from the column components. These sizes may be varied in other embodiments depending upon the particulars of a given implementation of system 100. In still other embodiments, one or more of tubing 510, segments 506 and 512 may be omitted or modified, such as where collection reservoir 106 is configured to directly interface with second end 112 of filter column 104.

In FIG. 5C, a fitting 514 for attaching vacuum pump 108 to collection reservoir 106 is depicted. A stopcock 516 may be equipped next to fitting 514 to help regulate suction coming from vacuum pump 108. In embodiments of system 100 where multiple arrangements of filter columns 104 and associated collection reservoirs 106 share a common vacuum pump 108, each stopcock 516 may enable the selective engagement of different filter columns 104.

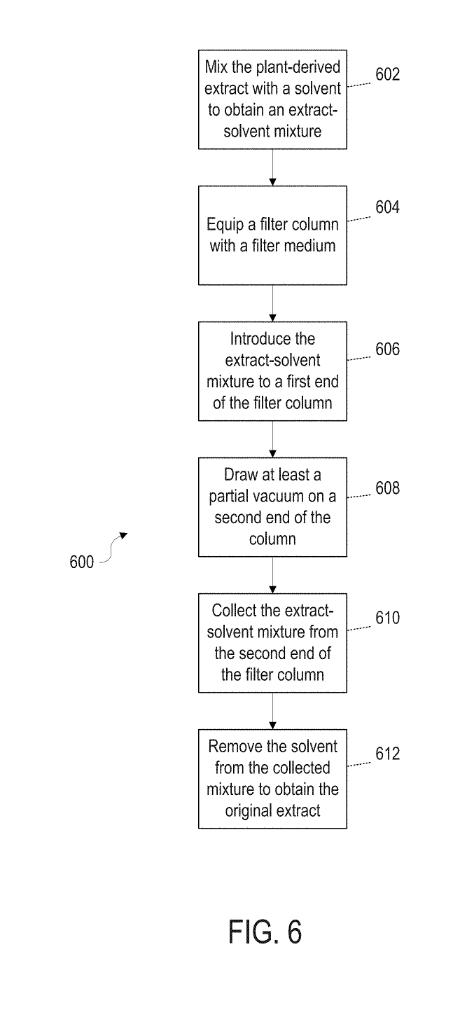

FIG. 6 depicts a possible method 600 for using system 100, according to various embodiments. Method 600 may be performed in whole or in part on system 100. Method 600 comprises mixing, in operation 602, the plant-derived extract with a solvent to obtain an extract-solvent mixture. The choice of solvent may be made with respect to a particular filter medium 202 employed within filter column 104. Some possible solvents may include ethanol, ethyl acetate, methanol, 2-propanol, 1-propanol, hexanes, heptanes, pentanes, ethyl ether, or methyl tert-butyl ether. The choice of solvent may also or alternatively be made with respect to a particular contaminant or contaminants that are targeted for removal. Moreover, the ratio of solvent to extract may be determined with respect to the given characteristics of a particular embodiment of system 100, a chosen filter medium 202 and solvent, as well as a desired flow or throughput of system 100. As mentioned above, a ratio in the range from 1:1 to 10:1 solvent to extract has been found to yield acceptable efficiency and performance in various embodiments. Other considerations may include the level of contaminants within the plant-derived extract, which may require a minimum or maximum flow rate to meet a desired level of contaminant removal.

Operation 604 includes equipping a filter column 104 with the filter medium 202. As discussed above, the filter medium 202 may be selected with respect to a given solvent to be mixed with the plant-derived extract, and vice-versa. The purification process may start in operation 606, which includes introducing the extract-solvent mixture to a first end 110 of the filter column 104. Purification may commence automatically where the extract-solvent mixture is drawn through filter column 104 via gravity feed.

For systems 100 that use a vacuum to either draw or assist drawing of the extract-solvent mixture through the filter column 104, operation 608 may be executed and includes drawing at least a partial vacuum on a second end 112 of the filter column 104, the second end 112 separated from the first end 110 by the filter medium 202, to cause the extract-solvent mixture to pass through the filter medium 202. In other possible embodiments, solution reservoir 102 may be pressurized to cause the extract-solvent mixture to be forced through filter column 104.

As the extract-solvent mixture passes through filter column 104, operation 610 includes collecting the extract-solvent mixture from second end 112 of the filter column 104. Following collection of the purified extract-solvent mixture, operation 612 includes removing the solvent from the extract-solvent mixture collected from the second end of the filter column to obtain the original plant-derived extract. The technique used for removal may depend upon the type of solvent mixed with the plant-derived extract. For example, some solvents may be removed by passing the purified mixture through a rotary evaporator. Additional or alternative post-filtration steps may be employed as necessary to effect removal of the solvent to a desired level.

As described above, system 100 is depicted in one possible embodiment. Various components of system 100 may vary without departing from the scope of this disclosure. For example, collection reservoir 106 may be configured as a single common reservoir that accepts feeds from multiple filter columns 104, such as through a manifold. Similarly, a single solution reservoir 102 may feed into multiple filter columns 104. Moreover, multiple collection reservoirs 106 may be chained together serially such that second and subsequent reservoirs are filled consecutively as previous reservoirs 106 fill.

Another possible variant can employ a pump to pressurize system 100 on the first end 110 of filter column 104, as opposed to applying a vacuum or suction to second end 112 of filter column 104. Such a configuration may be achieved by applying pressure into solution reservoir 102 above the extract-solvent mixture, to force it into tubing 114 (if present) and first end 110 of filter column 104. In some embodiments, vacuum pump 108 can be reconfigured or alternately configured to supply the necessary pressure (e.g. by using an exhaust port on the pump 108, if so equipped).

It should also be understood that method 600 may be repeated iteratively, but with different solvent/filter medium selections, if multiple contaminants are present that cannot be feasibly removed in a single pass with one selection of solvent/medium. Moreover, other configurations may be possible where multiple filter columns 104 are chained together (e.g. the second end 112 of a first filter column 104 feeds into a first end 110 of a second filter column 104, which may feed to others, etc.), to enhance purification or possibly to use a different filter medium 202 (possibly where the different medium is nevertheless compatible with the chosen solvent).

One possible extraction protocol that may be employed using system 100 and method 600 consists of six phases: A. Apparatus Assembly, B. Solution Preparation, C. Column Preparation, D. Sample Purification, E. Solvent Removal, and F. Apparatus Disassembly and Cleaning. The steps for the six phases are described below as a series of task lists.

A. Apparatus Assembly: System 100 may be assembled as follows:
1. Insert a filter disc 402 into the PTFE reducing coupling (50/25) (second fitting 206) so that its rough side will face the column 104 interior
2. Place an FETFE O-ring (size 136) 404 around the top of the second fitting 206, above its threads
3. Seat the second fitting 206 onto the bottom (second end 112) of a filter column 104 and hand-tighten
4. Affix the filter column 104 to a secure rack or frame, using two or three sets of clamps and/or clamp holders
5. Add approximately 0.5 kg of filtration media 202 to the column 104
6. Insert a filter disc 302 into the threaded PTFE adapter (#50) (first fitting 204) so that its rough side will face the column 104 interior
7. Place an FETFE O-ring (size 136) 304 around the bottom of the first fitting 204, below its threads
8. Seat the first fitting 204 onto the top (first end 110) of the filter column 104 and hand-tighten it
9. Insert a 75-100 mm segment of ⅜-in PTFE tubing horizontally into the opening of the PTFE tube compression elbow (⅜ npt) 306 and secure it by threading on the PVDF nut that comes with the elbow piece 306
10. Thread the compression elbow 306 onto the first fitting 204 into orifice 308 on the top of the column 104
11. Place an FETFE O-ring (size 121) 406 just below the threads of the second fitting 206 at the bottom of the column 104
12. Thread a 24/40 #25 glass conversion adapter 502 onto the second fitting 206
13. Insert a 60-100 mm segment of ⅜ in PTFE tubing 506 over the base of a 24/40 glass adapter 518 with stopcock 504
14. Using high vacuum grease, lightly lubricate the frosted part of the top of the glass adapter 518 with stopcock 504
15. Insert the glass adapter 518 with stopcock 504 into the larger glass conversion adapter 502, and give it a % turn to distribute the grease
16. Secure the connection between the two adapters with a 24/40 plastic glassware clip 520; orient the clip 520 so that the longer piece is above the shorter one
17. Insert a rubber stopper (fitting 508) that contains a 10-12 cm length of PTFE tubing 510 into the mouth of a vacuum filter flask (collection reservoir 106)
18. Place a 2-3 cm length of ¼-in vacuum tubing 512 over the top of the PTFE tubing 510 coming through the stopper
19. Insert a stopcock 516 into a piece of ¼-in vacuum tubing that is long enough to connect the filter flask 106 to where the vacuum pump 108 will be plugged in
20. Cover the other end of the stopcock 516 with a 4-cm length of ¼-in vacuum tubing
21. Connect the short length of vacuum tubing to the sidearm (fitting 514) of the filter flask 106, using an extra connector piece as needed to achieve a tight seal
22. Connect the long length of vacuum tubing to the vacuum pump 108
23. Place the flask 106 underneath the column 104 and carefully lower the column (releasing the labjaws clamps as needed) until the ⅜-in PTFE tubing 506 extending downward from the column 104 components covers the ¼-in vacuum tubing segment 510 that extends upward from the flask's rubber stopper.
24. Secure the labjaws clamps holding the column 104 and ensure that the column 104 is in a straight vertical position and not tilted in any direction
25. Secure the flask 106 to a rack or frame, using a labjaws clamp and clamp holder

B. Solution Preparation:
1. Place a funnel inside the neck of a 10-liter borosilicate glass media bottle (solution reservoir 102) and weigh the target amount of contaminated extract product into the bottle 102
2. The extract product is diluted with 5 times its weight with the appropriate Solvent. A typical solvent is ethanol for pesticide removals, or a straight chain hydrocarbon such as pentane, depending on the type of pesticide. As described above, in one example, the extract product may be a cannabis oil.
3. Dilute the product with 1 to 10 times its weight of the appropriate organic solvent, and transfer to solution reservoir/bottle 102
4. Cap the bottle 102 and fully dissolve the product by shaking vigorously or mixing with an overhead stirrer apparatus
5. Place the bottle 102 on a rack above the column 104
6. Replace the bottle's cap with one that has a drilled hole
7. Thread a piece of ¼-in vacuum tubing 114 through the cap that is long enough to reach both the bottom of the bottle 102 and the top (first end 110) of the column 104
8. Close off the vacuum tube 114 near the bottle 102 with a plastic tubing clamp
9. Proceed immediately to the Column Preparation phase

C. Column Preparation: Depending upon the composition of the filtration media 202, preparing the column 104 by thoroughly wetting it with solvent is essential for optimal pesticide extraction.
1. Close the stopcock 504 at the base of the column 104
2. Remove the adapter (first fitting 204) from the top 110 of the column 104
3. Carefully pour solvent into the column 104, up to where the column threads begin; repeat as needed to maintain this level of solvent
4. Start flow by: a) turning on the vacuum pump 108, b) opening the stopcock 504 between the vacuum pump 108 and the flask 106, and c) opening the stopcock 504 at the base 112 of the column 104
5. Add solvent to the top of the column 104 continuously, maintaining its level a couple of inches above the media 202 (do not let the media run dry)
6. When solvent is drawn about ¾ of the way down the column 104, stop flow by: a) closing the stopcock 504 at the base 112 of the column 104, b) closing the stopcock 504 between the flask 106 and the vacuum pump 108, and c) turning off the vacuum pump 108
7. Top off the column 104 with solvent a couple more times as needed
8. Put the adapter 204 back on top 110 of the column 104 and hand-tighten it
9. Proceed immediately to the Sample Purification phase.

D. Plant Extract Purification:
1. Use a solvent-filled plastic wash bottle to fill the segment of vacuum tubing 114 above the plastic tubing clamp with solvent
2. Attach the vacuum tubing 114 to the ⅜-in PTFE tubing 310 extending out of the compression elbow 306 on the top 110 of the column 104
3. Open the plastic tubing clamp
4. Start flow by: a) turning on the vacuum pump 108, b) opening the stopcock 516 between the vacuum pump 108 and the flask (collection reservoir) 106, and c) opening the stopcock 504 at the base 112 of the column 104
5. Duration of filtration is typically 50-80 minutes
6. Monitor the level of solution in the bottle (solution reservoir) 102 and tilt it as needed to keep the vacuum tubing 114 continuously submerged
7. Allow flow to continue until the solution is a few millimeters above the filtration media 202
8. Stop flow by: a) closing the stopcock 504 at the base 112 of the column 104, b) closing the stopcock 516 between the flask 106 and the vacuum pump 108, and c) turning off the vacuum pump 108
9. To recover solution that remains in the column 105, attach a length of ⅜-in vacuum tubing to the pressure outlet (as opposed to the suction port) of the vacuum pump 108 and connect it to the ⅜-in PTFE tubing 310 coming out of the compression elbow 306
10. Open the stopcock 516 on the vacuum tubing 114 that leads out of the vacuum pump's 108 inlet
11. Put on a mask (eye protection is already worn at all times) to prevent inhaling the liquid droplets or particulate matter that may be released
12. Firmly grasp the area where the vacuum tubing 114 covers the ⅜-in PTFE tubing 310
13. Open the stopcock 504 at the base 112 of the column 104
14. Turn on the vacuum pump 108
15. Maintain a firm grasp on the vacuum tubing 114 connection until solution ceases to elute from the column 104
16. Turn off the vacuum pump 108 and remove the vacuum tubing 114 from the top 110 of the column 104
17. Detach the flask 106 from the column 104 and vacuum pump 108
18. The next phase, Solvent Removal, does not need to be performed immediately. The filtered solution is stable for up to seven days at slightly below room temperature or up to 30 days at −20° C. The solution should, however, be stored in a sealed glass container to prevent evaporation or spillage.

E. Solvent Removal: To restore the extracted product's original viscosity and composition, the solvent used to dissolve the product into solution must be removed. This phase also ensures that the finished product will pass regulatory compliance testing for residual solvents. Solvent removal is done with an industrial scale rotary evaporator that ideally has an evaporator flask with a capacity of 20 liters. There are many suitable models of rotary evaporators and all must be used in combination with a vacuum pump and a chiller of appropriate specifications. The steps outlined below describe the general solvent removal process, rather than specific steps for any particular model of rotary evaporator.
1. Turn on the chiller and allow it to reach a target temperature of −12-24° C.
2. Fill the bath with fresh water and select the target temperature for the heater (which will be determined based on which solvent is being removed)
3. Add the filtered solution to the rotary evaporator's evaporator flask until the flask is just over half full
4. Set a target value for the vacuum and begin rotation of the flask
5. Monitor the appearance and movement of the solution in the evaporator flask
6. Monitor the accumulation of solvent in the collection flask and empty it as needed
7. Ethanol may need to be added to the evaporator flask one or more times, to facilitate the removal of the primary solvent; this should be done with the water bath temperature set at 64° C.
8. Gradually increase the vacuum (ultimately down to 0 mbar) while lowering the rotation speed of the evaporator flask, to ensure removal of all solvent residue
9. Once solvent has ceased to drip from the evaporation coil, the process is complete
10. Release the vacuum
11. Transfer the oil from the evaporator flask into a tared glass receptacle
12. Remove solvent from the collection flask
13. Clean the evaporator flask
14. Power down all equipment

F. Apparatus Disassembly and Cleaning: After the filtration process is done, the following cleaning should be performed within a few hours.
1. Set the filter flask (collection reservoir) 106 aside for the pending Solvent Removal phase
2. Remove the glassware clip 520, glass conversion adapter 502, glass adapter 518 with stopcock 504, and FETFE O-ring (size 121) 406 from the bottom (second end) 112 of the column 104 and disassemble the glass adapter with stopcock 504
3. Use a solvent wash bottle to rinse the individual pieces over a solvent waste container, to dissolve any extracted product residue
4. Repeat step 3 with ethanol to rinse off the previous solvent
5. Soak the components in clean ethanol overnight
6. Place a waste media receptacle underneath the column 104
7. Remove the coupler (second fitting) 206 from the column 104
8. Attach a length of ⅜-in vacuum tubing to the pressure outlet of the vacuum pump 108 and connect it to the ⅜-in PTFE tubing 310 coming out of the compression elbow 306 (this step and the seven that follow were done previously, following filtration, to purge solution that remained in the column)
9. Open the stopcock on the vacuum tubing that leads out of the vacuum pump's 108 inlet
10. Put on a mask (eye protection is already worn at all times) to prevent inhaling the particulate matter that may be released
11. Firmly grasp the area where the vacuum tubing covers the ⅜-in PTFE tubing 310
12. Open the stopcock 504 at the base 112 of the column 104
13. Turn on the vacuum pump 108
14. Maintain a firm grasp on the vacuum tubing connection until used media ceases to exit the column 104
15. Turn off the vacuum pump 108 and remove the vacuum tubing from the top 110 of the column 104
16. Remove and seal the waste receptacle
17. Remove the adapter (first fitting) 204 with attached compression elbow 306 from the top 110 of the column 104
18. Remove and discard the first and second filter discs 302 and 402 from both the adapter 204 and the coupler (second fitting) 206
19. Remove the two FETFE O-rings (size 136) 304 and 404 from the adapter 204 and coupler 206, respectively, rinse them with solvent, and soak them in ethanol overnight
20. Disassemble the compression elbow 306, rinse the components with solvent, and then soak them overnight in ethanol
21. With the column 104 still secured on the rack and with a solvent waste receptacle beneath it, use a solvent wash bottle to rinse the column until all visible media is removed
22. Rinse the column 104 three more times and then allow it to air dry

What is claimed is:

1. An apparatus for purification of a plant-derived extract, comprising:
a filter column with a first end and a second end;
a filter medium contained within the filter column;
an extract-solvent mixture solution reservoir in fluid communication with the first end; and
an extract-solvent mixture collection reservoir in fluid communication with the second end;
wherein a solvent of the extract-solvent mixture and the filter medium are selected to optimize removal of at least one contaminant from the plant-derived extract.

2. The apparatus of claim 1, further comprising a vacuum pump configured to draw at least a partial vacuum, the vacuum pump in fluid communication with the second end.

3. The apparatus of claim 2, wherein the vacuum pump is connected to the collection reservoir.

4. The apparatus of claim 1, further comprising a first filter disc in the first end and a second filter disc in the second end, the first and second filter discs configured to prevent escape of the filter medium.

5. The apparatus of claim 4, wherein the first and second filter discs each have a plurality of perforations approximately 20 μm in diameter.

6. The apparatus of claim 1, wherein the plant-derived extract is an oil or resin obtained from a cannabis plant.

7. The apparatus of claim 1, wherein the at least one contaminant is an insecticide, fungicide, or herbicide, heavy metal or other foreign organic containments.

8. The apparatus of claim 1, wherein the filter medium is at least one of a silica bonded functional group (i.e. C8 media) medium, reverse phase resin, de-ionization resin, activated magnesium silicate, activated carbon, or silica gel.

9. The apparatus of claim 1, wherein the collection reservoir comprises a single or plurality of collection reservoirs.

10. The apparatus of claim 1, further comprising a stopcock between the second end and the collection reservoir, the stopcock configured to control a flow of extract-solvent mixture through the filter column or other valve to stop flow.
